(19) United States
(12) Patent Application Publication
(10) Pub. No.: US 2012/0277723 A1
(43) Pub. Date: Nov. 1, 2012

(54) SYSTEM AND METHOD FOR THE INTEGRATION OF FUSED-DATA HYPOGLYCAEMIA ALARMS INTO CLOSED-LOOP GLYCAEMIC CONTROL SYSTEMS

(75) Inventors: Victor Skladnev, Vaucluse (AU); Stanislav Tarnavskii, Allawah (AU); Thomas McGregor, West Ryde (AU); Nejhdeh Ghevondian, Ryde (AU)

(73) Assignee: AiMedics Pty Ltd, Eveleigh (AU)

(21) Appl. No.: 13/504,698

(22) PCT Filed: Nov. 4, 2010
(86) PCT No.: PCT/AU2010/001467

§ 371 Date: Jul. 13, 2012

(30) Foreign Application Priority Data
Nov. 4, 2009 (AU) 2009905385

(51) Int. Cl. A61M 5/168

(52) U.S. Cl. 604/504; 604/66

(57) ABSTRACT

Methods and systems are described for controlling a flowrate of insulin infused into the body of a patient. An insulin infusion device infuses insulin into the body of the patient. A first sensor generates blood glucose level (BGL) data indicative of a blood glucose level of the patient. A second sensor generates autonomic nervous system (ANS) data such as heart rate data dependent on at least one parameter of the patient's autonomic nervous system. A data fusion processor receives the BGL data and the ANS data and generates an output alarm signal if a hypoglycaemic event is inferred. A flowrate of insulin of the insulin infusion device may be modified dependent on the output alarm signal.

### FIELD OF THE INVENTION

The present invention relates to closed-loop glycaemic control systems and in particular to the integration of safety features into the control system.

### BACKGROUND OF THE INVENTION

Landmark studies have demonstrated the efficacy of tight glucose control in the prevention of long term complications of diabetes (See, for example the Diabetes Control and Complications Trial Research Group report on “The effect of intensive treatment of diabetes on the development and progression of long-term complications in insulin-dependent diabetes mellitus.” N Engl J Med. 1993;329:977-986 and the UK Prospective Diabetes Study (UKPDS) Group: “Intensive blood-glucose control with sulphonylureas or insulin compared with conventional treatment and risk of complications in patients with type 2 diabetes.” Lancet. 1998;352:836-853.)

Despite this, a high proportion of diabetics do not achieve recommended glycaemic targets. For many diabetics the near-term fear of undetected hypoglycaemia is a barrier to achieving tight glucose control in practice. Advances in the development of continuous glucose monitoring systems (CGMS) have offered a major potential to improve diabetes care through integration in closed-loop glycaemic control systems. General implementation of closed-loop control systems however has been constrained by the lack of reliably accurate hypoglycaemia alarm systems. While generally accurate, at low glucose levels CGMS suffer from significant noise due in part to calibration offset effects and drift. The implementation of closed-loop systems for glycaemic control is thus limited by safety concerns from the possible serious or even fatal consequence of closed-loop systems based on CGMS continuing to infuse insulin under hypoglycaemic conditions. This limitation is particularly significant when the user is asleep. During sleep hypoglycaemia awareness is compromised, resulting in a low probability of the user being able to independently take corrective action.

In these circumstances there is a need for a glycaemic control system that is sensitive to hypoglycaemia whilst maintaining an acceptable false-positive alarm rate.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

The object of this invention is to overcome or at least ameliorate one or more of the problems with prior art systems.

Disclosed herein is a system for fusing two data sources that are substantially statistically independent, in order to generate critical safety outputs including infusion-cut-off signals to control insulin pumps or like devices. In one arrangement one of the data sources is derived from a continuous glucose monitoring system (CGMS) and the other from autonomic nervous system (ANS) data.

According to a first aspect of the invention there is provided a system for controlling a flowrate of insulin infused into the body of a patient, the system comprising:
an insulin infusion device that in use infuses insulin into the body of the patient;
a first sensor that in use generates BGL data indicative of a blood glucose level of the patient;
a second sensor that in use generates ANS data dependent on at least one parameter of the patient's autonomous nervous system; and
a processor that receives the BGL data and the ANS data and, based on the received data, generates an output alarm signal if a hypoglycaemic event is inferred; and
a controller that modifies a flowrate of insulin of the insulin infusion device dependent on the output alarm signal.

In broad terms the invention relates to a system for fusing two data sources that are substantially statistically independent, in order to generate an infusion-cut-off signal to control an insulin pump. One of the data sources may be derived from a continuous glucose monitoring system (CGMS) and the other from autonomic nervous system (ANS) data.

According to a further aspect of the invention there is provided a method for monitoring a flowrate of insulin infused into the body of a patient by an insulin infusion device, the method comprising:
receiving BGL data indicative of a blood glucose level of the patient;
receiving ANS data dependent on at least one parameter of the patient's autonomous nervous system;
maintaining an ANS-difference signal data based on a difference between the ANS data and a time-lagged version of the ANS data;
triggering a first intermediate alarm if the BGL data indicates a hypoglycaemic event;
triggering a second intermediate alarm if the ANS-difference signal indicates a hypoglycaemic event;
outputting an alarm signal to the insulin infusion device if the first intermediate alarm and the second intermediate alarm are triggered.

The invention also resides in instructions executable by a data fusion processor to implement a method of analysing BGL data and ANS data and to such instructions when stored on a storage medium readable by the data fusion processor.

As used herein, except where the context requires otherwise, the term “comprise” and variations of the term, such as “comprising”, “comprises” and “comprised”, are not intended to exclude further additives, components, integers or steps.

### BRIEF DESCRIPTION OF THE DRAWINGS

Embodiments of the invention are described below with reference to the drawings, in which:

FIG. 1 is a schematic block diagram of a closed-loop glycaemic control system that fuses data from a blood glucose monitor and a monitor measuring data pertaining to the patient's autonomous nervous system (ANS);

FIG. 2A is a schematic diagram of a chest-belt transmitter that may be used in the implementation of the present invention;

FIG. 2B is a schematic diagram of a receiver unit that may be used in conjunction with the transmitter of FIG. 2A;

FIG. 3 is a flow diagram of a method for monitoring a user's ANS data and triggering an alarm if a hypoglycaemia event is detected;

FIG. 4 is a flow diagram of a method for monitoring and processing ANS data and blood glucose level (BGL) data; and

FIG. 5 is a flow chart of a method of fusing BGL and ANS data to detect hypoglycaemic events in a patient using a closed-loop insulin infusion system.

### DETAILED DESCRIPTION OF EMBODIMENTS

The methods and systems described herein aim to provide solutions to the problem of closed-loop glycaemia control in circumstances wherein the continued infusion of insulin or another therapeutic agent could cause serious injury or death. The described method uses the fusion of CGMS blood glucose level/trend data with information pertaining to the patient's autonomic nervous system to provide a critical alarm function.

This critical alarm function is integrated into the closed-loop system to modify (for example, to stop) continued infusion under conditions where the user's blood glucose levels are lower than desirable, without significantly altering the incidence of false alarms.

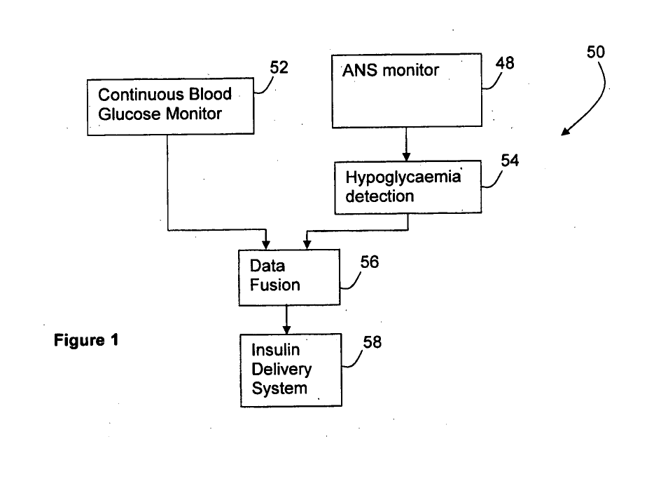

FIG. 1 is a schematic diagram of a glycaemic control system 50. A continuous glucose monitoring system (CGMS) 52 measures the patient's blood glucose level (BGL) on a regular basis. Such monitors are commercially available from suppliers including Medtronic and typically consist of a disposable sensor positioned under the patient's skin and regularly replaced. An output signal from the CGMS 52 is communicated to a receiver unit that displays and further processes the BGL measurement. The CGMS 52 typically provides readings once every five minutes or once every minute.

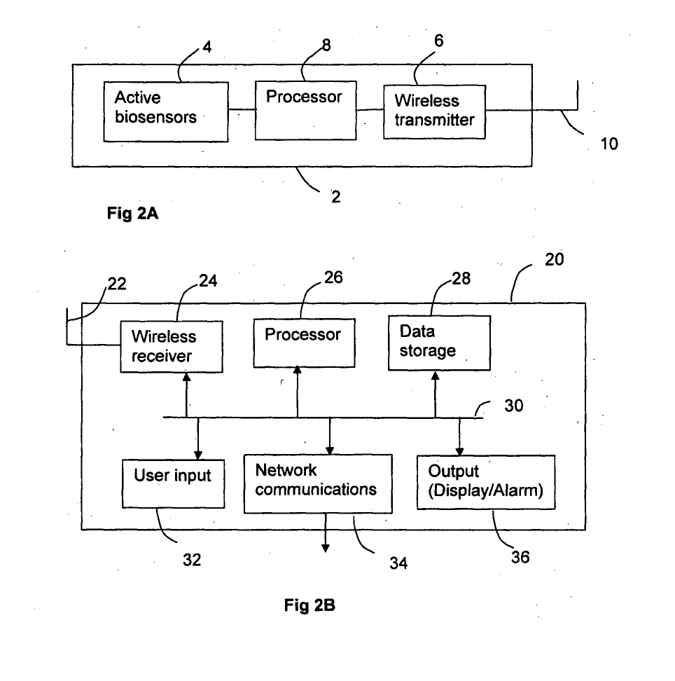

A monitor 48 measures information pertaining to the patient's autonomous nervous system (ANS). This data includes the patient's heart rate. The output from the ANS monitor 48 is processed by a module 54 that detects hypoglycaemic events. An example of an ANS monitor 48 and hypoglycaemic detection module 54 is described below with reference to FIGS. 2A and 2B.

The outputs of the CGMS 52 and the hypoglycaemia detection module 54 are processed in a data fusion module 56 to provide an alarm function if a hypoglycaemic event is detected. The hypoglycaemia detection module 54 and data fusion module 56 may be implemented on a common processing platform or they may be implemented in distributed units.

An insulin delivery system 58 infuses insulin into the patient. Insulin pumps are available commercially and typically include a reservoir for holding a supply of insulin, a cannula for subcutaneous positioning, a pump and a control module. The BGL data and the output of the data fusion module 56 are communicated to the insulin delivery system 58, which uses the input data to control infusion of insulin into the patient.

In one arrangement the control of the insulin delivery system 58 is a cut-off signal when the data fusion module 56 indicates a critical alarm. In other arrangements the flow of insulin may be continually varied dependent on the monitored data. For example, provided no hypoglycaemia event is detected, the insulin delivery system 58 may determine the insulin flow based on deviations from desired BGL setpoints. Proportional, integral and/or derivative (PI/PID) controllers may use inputs derived from the fused data in a manner known to control system specialists. Other control approaches may also be used, for example model predictive approaches that employ models of the patient's response to insulin.

The closed-loop control of insulin may be supplemented by feed-forward methods where other sources of information are available. For example, the patient may notify the insulin delivery system 58 that he or she is about to eat and the control algorithm may increase the delivery of insulin prior to the meal. Likewise, information on relevant features such as time of day and exercise may be utilised.

Data communication between the CGMS 52, ANS device 48, the platform supporting the modules 54, 56 and the infusion system 58 such as insulin pumps may be via wire, fibre optics, RF links or similar systems. In other embodiments, these components may be incorporated into combined units.

FIG. 2A illustrates an example of an ANS monitor. In this arrangement, a patient may wear a chest-belt unit 2 which, in use, is located around the patient's upper thoracic region. The chest-belt unit 2 may have an adjustable elasticated strap which is adapted to engage tightly around the patient's chest. A suitable and secure fastening system which is relatively easy to engage and disengage enables the belt unit 2 to be put on and taken off without difficulty. The strap can also be adapted to fit around a child's chest in the same manner as an adult patient. The belt unit 2 incorporates an electronic housing that encloses a wireless transmitter, analogue electronic circuitry and a microcontroller.

As shown in FIG. 2A, the belt unit 2 includes active biosensors 4 that may be skin surface electrodes each adapted to monitor a different physiological parameter. The sensors 4 measure physiological parameters such as skin impedance, ECG and segments thereof, including QT-interval and ST-segment, heart rate and the mean peak frequency of the heart rate. These aspects are further discussed in PCT/AU02/00218, published as WO 02/069798. The sensors and signal processing systems preferably have sufficient sensitivity and accuracy to enable extraction of subcomponents of the ECG such as the QT interval.

The biosensors 4 provide the signals which, after being processed, amplified, and filtered by analogue electronic circuitry, are interfaced to the processor 8, which may be a microcontroller (μC) unit. The μC unit 8 digitises the signals using an A/D (analogue-to-digital) converter and provides the digitised signals to a wireless transmitter 6 with an aerial 10.

Associated with the belt unit 2 is a receiver unit 20 which is adapted to process signals monitored by the unit 2 for analysis and alarms. The hypoglycaemia detection module 54 may be implemented as software running on the receiver unit 20. The data fusion module 56 may also run on the receiver unit 20.

The units 2 and 20 may be encoded to recognise one another for secure communication. As shown in FIG. 2B, the receiver unit 20 has an aerial 22 and wireless receiver 24. Data may be stored in data storage 28 and processed by software running on the processor 26. Data communication between the components of the receiver unit 20 is provided by bus 30. The unit 20 may have one or more output units 36 including a display for displaying information to the user. The outputs 36 may also include an audible alarm.

A network communication interface 34 may also be included. This permits information about the patient's physiological condition to be transmitted elsewhere, for example via an Internet connection to a health-care provider such as an endocrinologist or cardiologist. In another example information may be sent via an SMS messaging service. Thus, for example, if the units 2, 20 are monitoring a child, a message may be sent to the child's parents if an alarm is triggered. Output signals from the receiver unit 20 are provided to the insulin delivery system 58, for example via an RF link or a fibre optic connection. Alternatively, the receiver unit 20 may be integrated with the insulin 25. delivery system 58.

The unit 20 may also include a user input 32 that permits additional information to be entered into the unit 20. For example, if the patient takes a reading of blood glucose level (BGL) using a finger-prick device, the result may be entered into the unit 20 using a keypad. Alternatively or additionally, the input 32 may be a data link to other equipment such as the CGMS 52 or finger-prick device.

An example of a suitable monitoring system is the HypoMon described in patent application WO 2004/098405 titled “Patient Monitor”.

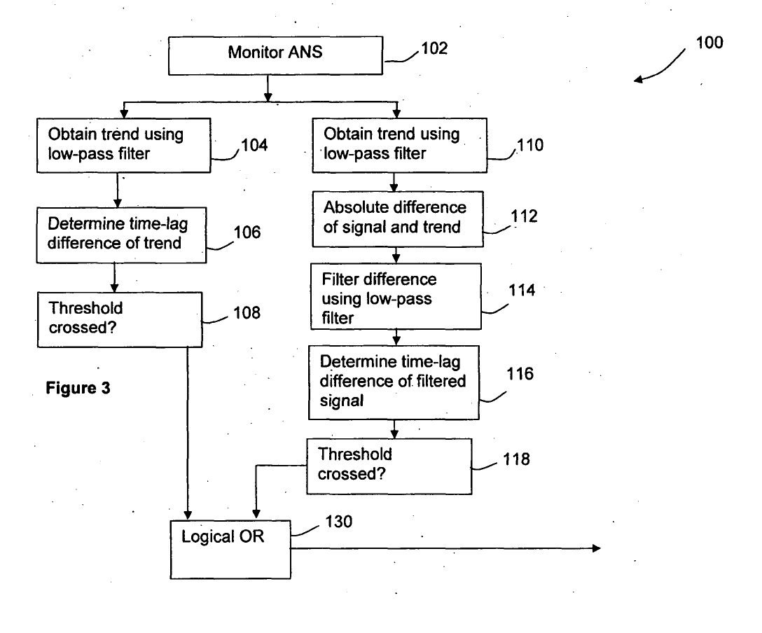

A method 100 for monitoring ANS data to detect a hypoglycaemia event is shown in FIG. 3. A patient's ANS data, including heart rate, is monitored (step 102), for example using the unit 2 described with reference to FIGS. 2A and 2B.

In method 100, the ANS data, such as heart rate data, is analysed in two different ways (steps 104-108 and 110-118 respectively) and the results are combined to trigger an alarm if appropriate. The steps 104-130 of method 100 may be performed by software running on the processor 26 of the receiver unit 20. It will be appreciated that the method 100 may have different implementations. For example, information may be forwarded from the unit 20 to a remote server for processing. The method 100 could also be performed in a distributed fashion, where different portions of the method are carried out using different processors. The method 100, or parts of the method 100, may also be performed using other processing means such as analog circuitry, application-specific integrated circuits (ASICs) or field-programmable gate arrays (FPGAs).

### Time-Lag Trend

In step 104 the patient's ANS signal data is passed through a low-pass filter to obtain a low-frequency trend as a function of time. In one arrangement the filter has a time constant of 1.6 hours. Methods of selecting parameter values for the method 100 will be discussed below.

In step 106 a time-lag trend is determined. The time-lag trend is a function of time calculated as a difference between a value of the low-frequency trend at time t=i and a past value of the low-frequency trend at time t=(i−T_{lag}). In the inventor's view step 106 is a normalizing process that establishes a dynamic baseline for the process before the occurrence of hypoglycaemia. The time-lag trend monitors the change in ANS signal (eg heart rate) with respect to the dynamic baseline.

In step 108 the monitoring software checks whether a specified threshold has been crossed. The triggering event may correspond to a drop in the patient's BGL.

### Difference Between ANS Signal and ANS Trend

Steps 110-118 represent another analysis of the input ANS signal data. In step 110 the ANS data is filtered using a low-pass filter to provide a low-frequency trend. In one implementation the time constant of the filter is 0.3 hours. Then, in step 112, the absolute difference between the raw ANS (heart-rate) data and the low-frequency trend is determined. A delayed version of the raw data may be used when determining the absolute difference. The delay may be selected to match the delay inherent in the low-pass filtering of step 110.

The absolute difference signal is then processed in a similar way to the method of steps 104-108. That is, steps 114, 116 and 118 correspond to steps 104, 106 and 108, although the parameters used in processing may differ.

In step 114 the absolute difference signal is passed through a low-pass filter to obtain a low-frequency difference trend. In one arrangement the filter has a time constant of 2.1 hours.

In step 116 a time-lag trend is determined as a difference between a value of the low-frequency difference trend at time t=i and a past value of the trend at time t=(i−T_{lag}). The time T_{lag} need not be the same as the lag time used in step 106. In one arrangement the T_{lag} for step 116 is 2.1 hours. Then, in step 118, the monitoring software checks whether the output signal from step 116 crosses a specified threshold. If so, an intermediate flag is triggered.

The thresholds used in steps 108 and 118 may differ from one another.

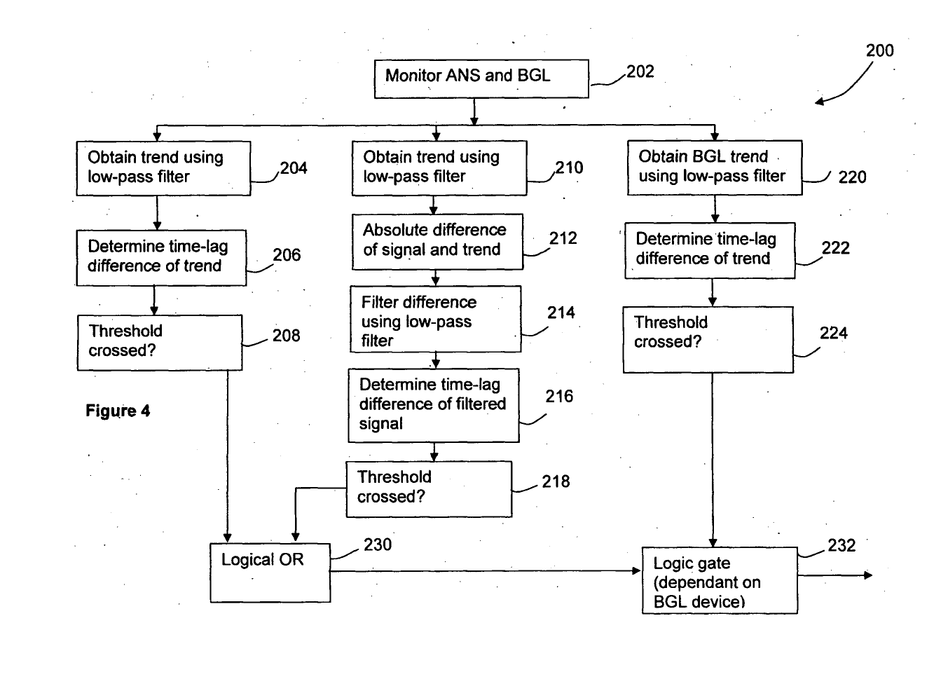

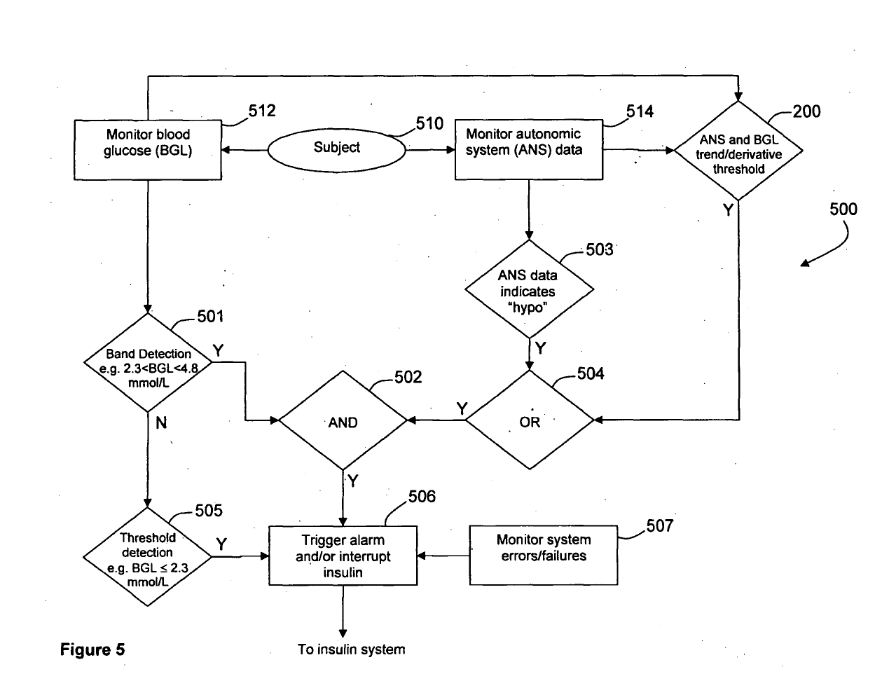

The alarm method 100 combines the outputs of steps 108 and 118. Step 130 is a logical OR operation. If step 108 detects a threshold crossing OR step 118 detects a threshold crossing, then the logical OR of step 130 triggers a further flag, which is indicative of a potential hypoglycaemic event. The flag may be used in further processing, for example in the methods illustrated in FIGS. 4 and 5. Alternatively or in addition, an intermediate alarm may be emitted by the receiver unit 20 if the logical OR 130 triggers the flag. For example, an audible alarm may be sounded, or a message may be transmitted to a carer to indicate potential hypoglycaemia. The alarm may also be provided to the data fusion module 56 as described in more detail with reference to FIG. 5.

Test results obtained by the inventors suggest that method 100 provides an alarm for overnight hypoglycaemia events based on ANS trend differences. The algorithm structure has inter-subject stability.

The structure of method 100 may be summarized as follows:

α(alarm)=β[[T(a) OR T(b)] AND Ψ[T(c)]]

where: T (a) is the response time of the time-lagged difference of the low pass filter components of ANS signal (low pass filter time constant 1.6 hours and lag 1.6 hours), eg steps 104-108;

T (b) is the response time of the absolute difference between ANS feature, e.g. heart rate, and ANS trend with a 0.3 hour time constant which is further converted to a trend difference as in T (a) where the filter time constant is 2.1 hours and the lag is 2.1 hours, eg steps 110-118;

T (c) is an optional function that is similar to T (b) but which focuses on higher frequency information. In one arrangement T (c) varies from T (b) in that the final low-pass filter has a time constant of 0.17 hours and a lag of 0.17 hours. The time window for the associated AND function may be 1.2 hours, ie if the two inputs to the AND function are triggered within a 1.2 hour window, the output of the AND is triggered. T (c) may be implemented as a series of operations similar to steps 110-118, but with parameters selected to consider higher-frequency information.

### Selecting Parameter Values

The method 100 includes several parameters, including time-constants for the low pass filters, lag times for calculating the lagged signals and the values of the thresholds used in steps 108 and 118. These parameters may be set by accumulating patient data including information about the onset of hypoglycaemia and dividing the data into training data sets and test data sets. The parameter values may be determined by training algorithms that optimize the values based on the training sets. The optimized parameter values may be tested on the test data sets. Such procedures may serve to increase the detection accuracy of the method and to reduce the number of false alarms.

One method for identifying stable signatures within the complex system nature of type-1 diabetes mellitus (T1DM) sufferer's response to hypoglycaemia was as follows. Selected non-invasive physiological parameters along with regular venous BGL readings on gold standard (YSI) devices were monitored on 130 T1DM volunteers over a range of day/night hypoglycaemic clamp and natural conditions. Analysis of this data was guided by the hypothesis that hypoglycaemia events stimulate physiological responses which show frequency, time-lag and time-window features that have inter-subject stability. Stability evaluations on potential features were then carried out in an iterative manner by segregating the data into training and evaluation data sets. The stability of the discovered signatures was then confirmed in a blinded prospective overnight trial on 52 previously unseen T1DM sufferers. Other subsystems are trained similarly.

A method 200 for monitoring ANS and BGL data to detect a hypoglycaemia event is shown in FIG. 4. A patient's ANS and BGL are monitored (step 202), for example using the units 2, 20 described with reference to FIGS. 2A and 2B and module 52 described with reference to FIG. 1. In method 200, the ANS features, such as heart rate, are analysed in two different ways (steps 204-208 and 210-218 respectively) and BGL is processed in steps 220-224, and the results are combined in operations 230 and 232 to trigger an intermediate alarm if appropriate. The steps 204-232 may be performed by software running on the processor 26 of the receiver unit 20. It will be appreciated that the method 200 may have different implementations. For example, information may be forwarded from the units 20 and 52 to a remote server for processing. The method 200 could also be performed in a distributed fashion, where different portions of the method are carried out using different processors. The method 200, or parts of the method 200, may also be performed using other processing means such as analogue circuitry, application-specific integrated circuits (ASICs) or field-programmable gate arrays (FPGAs).

### Time-Lag Trend

In step 204 the patient's ANS signal is passed through a low-pass filter to obtain a low-frequency ANS trend. In one arrangement the filter has a time constant of 1.6 hours. Methods of selecting parameter values for the method 200 are similar to those discussed above in the context of process 100 (FIG. 3).

In step 206 a time-lag trend is determined as a difference between a value of the trend at time t=i and a past value of the trend at time t=(i−T_{lag}). In the inventor's view step 206 is a normalizing process that establishes a dynamic baseline for the process before the occurrence of hypoglycaemia. The time-lag trend monitors the change in ANS trend with respect to the dynamic baseline.

In step 208 the monitoring software checks whether a specified threshold has been crossed. The triggering event may correspond to a drop in the patient's BGL.

### Difference Between ANS Signal and ANS Trend

Steps 210-218 represent another analysis of the input ANS data. In step 210 ANS signal is filtered using a low-pass filter, to provide a low-frequency trend. In one implementation the time constant of the filter is 0.3 hours. Then, in step 212, the absolute difference between the raw ANS data and the low-frequency trend is determined. A delayed version of the raw data may be used when determining the absolute difference. The delay is selected to match the delay inherent in the low-pass filtering.

The absolute difference signal is then processed in a similar way to the method of steps 204-208. That is, steps 214, 216 and 218 correspond to steps 204, 206 and 208, although the parameters used in processing may differ.

In step 214 the absolute difference signal is passed through a low-pass filter to obtain a low-frequency difference trend. In one arrangement the filter has a time constant of 2.1 hours.

In step 216 a time-lag trend is determined as a difference between a value of the low-frequency difference trend at time t=i and a past value of the trend at time t=(i−T_{lag}). The time T_{lag} need not be the same as the lag time used in step 206. In one arrangement the T_{lag} for step 216 is 2.1 hours. Then, in step 218, the monitoring software checks whether the output signal from step 216 crosses a specified threshold. If so, an intermediate flag is triggered.

Steps 220-224 represent a strand of processing of BGL data. Steps 220-224 correspond to the steps 204-208 but may use a different frequency pass-band.

In step 220 the BGL data is filtered using a low-pass filter to provide a low-frequency trend. In one implementation the time constant of the filter is 0.3 hours.

In step 222 a time-lag difference of trend is determined as a difference between a value of the second low-frequency difference trend at time t=i and a past value of the trend at time t=(i−T_{lag}). The time T_{lag} need not be the same as the lag time used in step 206 or 216. In one implementation the time T_{lag} of step 222 is equal to 0.3 hours. In step 224, the monitoring software checks whether the output signal from step 222 crosses a specified threshold. If so, an intermediate flag is triggered.

The thresholds used in steps 208, 218 and 224 may differ from one another.

The alarm method 200 combines the outputs of steps 208, 218 and 224. Step 230 is a logical OR operation. If step 208 detects a threshold crossing OR step 218 detects a threshold crossing, then the logical OR of step 230 triggers a further intermediate flag, which is provided to the logic gate of step 232. The other input to the logic gate is the output of step 224. From the logic gate 232 the intermediate alarm is provided to the data fusion module 56 as described in more detail with reference to FIG. 5.

The structure of method 200 may be summarized as follows:

α(alarm)=γ([T(a) OR T(b)], Ψ[T(c)])

where: T(a) is the response time of the time-lagged difference of the low pass filter components of ANS data (low pass filter time constant 1.6 hours and lag 1.6 hours);

T(b) is the response time of the absolute difference between ANS features, e.g. heart rate and heart rate trend with a 0.3 hour time constant which is further converted to a trend difference as in T (a) where the filter time constant is 2.1 hours and the lag is 2.1 hours;

T(c) is the response time of the time-lagged difference of the low pass filter components of BGL data (low pass filter time constant 0.3 hours and lag 0.3 hours).

The structure of the combination operation 232 may be dependent on the particular CGMS used to measure blood glucose, and may for example reflect a level of confidence in the CGMS output in different ranges.

### Using Dynamic Parameter Settings

The alarm thresholds and parameters such as decision integration times used in the described methods may be fixed or dynamic depending on the nature of the additional information available. For example, the measurements of blood glucose levels (BGL) from the continuous glucose monitor 52 may be integrated into the alarm system in the form of a logic tree of the following general form:

a) At high BGL values ignore all alarms over a specified time window;

b) At near-normal BGL values raise the threshold of alarm features;

c) At low BGL values or in the-event of significant trends to low BGLs lower the alarm thresholds for selected features; and d) At very low BGL estimates activate the alarm.

In this manner allowances may be made for variations in estimation accuracy over BGL ranges.

Thus, for example, the threshold levels in steps 208 and 218 may be raised or lowered dependent on the BGL or the BGL trend.

Alternatively, instead of adjusting the thresholds, scaling factors may be used to take additional information into account. For example, with reference to FIG. 4, a scaling factor may be applied to one or more of the trends before checking whether the trends have crossed the specified threshold (e.g. in steps 208 or 218). Thus, a scaling factor may be used as a multiplier for the time-lag difference obtained in step 206, and/or the time lag difference determined in step 216.

For example, direct estimates of blood glucose levels (BGL) and trends from a continuous glucose monitor may be integrated into the alarm system in the form of a logic tree of the following general form:

a) At high BGL estimates, ignore all alarms over a specified time window;

b) At near-normal BGL estimates, reduce one or more of the scaling factors to reduce the probability of the scaled trend exceeding the specified threshold;

c) At low BGL estimates or in the event of significant trends to low BGLs, increase one or more of the scaling factors to increase the probability of the scaled trend exceeding a specified threshold; and

d) At very low BGL estimates activate the alarm.

In this manner allowances may be made for variations in estimation accuracy over BGL ranges. The scaling coefficients may be varied dependent on the BGL value at the beginning of the night or on the history of BGL from the beginning of the night through to the latest reading.

### Data Fusion

FIG. 5 shows an example of a data fusion method 500 that may be used in the control system 50.

The combination of the complementary BGL and ANS parameters enables compensation for calibration and drift errors that may not be achievable through the manipulation of data derived from a single source such as blood glucose levels and rates of change. Clinical analyses indicate that when the two data sources are fused in an appropriate manner the information from each stream complements the other. In the method 500, the inventors propose that ANS signatures of hypoglycaemia are largely independent of CGMS data and hence may detect hypoglycaemia even if calibration and drift errors are large for the blood glucose measurement. CGMS data on the other hand may be used to reduce ANS-signature false alarms when measured blood glucose levels are well above the BGL device's error band.

In operation 512 of method 500 the CGMS 52 monitors the blood glucose level of the subject 510 on a regular basis. In process 501 the system checks whether or not the measured BGL is within a specified range of values. In one arrangement the range is from 2.3 to 4.8 mmol/L. The checking step 501 may be implemented at various points of the control system 50, for example within the monitor 52 or in the data fusion module 56. If the BGL measurement is within the designated range (the Y option of the checking step), then an intermediate alarm output is triggered and is input to the logical AND block 502. In effect, method 500 takes ANS data into account while the measured BGL is in the specified range.

In parallel to steps 501 and 502, in process 514 the ANS monitor 48 tracks data such as. heart rate of the subject 510. The ANS data generated in process 514 is analysed in step 503 to assess whether there is a current or immanent hypoglycaemic event. Step 503 may be implemented in the detection module 54 using, for example, the trend analysis method of FIG. 3. For example, steps 104 to 130 may be applied to the ANS data generated by the ANS monitor 48.

If the ANS data indicates a hypoglycaemic event (the Y output of process 503), an intermediate alarm signal is triggered and provided to the OR block 504, which may be implemented in the data fusion module 56.

The AND block 502 receives outputs from processes 501 and 504. If the band detection 501 and the ANS data through modules 503 and 504 indicate a hypoglycaemic event (the Yes output of the AND block 502) an alarm output may be triggered. In one arrangement the alarm output of 501 is constrained to operate only if the measured blood glucose level is between specified values, for example 4.8 and 2.3 mmol/L (86.4 and 41.1 mg/dL). This specified range may be determined heuristically and reflects calibration errors that have been noted in integrated CGMS system. Generally, accuracy is lower in the hypoglycaemic range than in the euglycaemic and hyperglycaemic ranges. The monitors become less accurate and more prone to drift at lower values of blood glucose. The performance of glucose monitors has been studied, for example in Wentholt I M, “Comparison of a Needle-Type and a Microdialysis Continuous Glucose Monitor in Type 1 Diabetic Patients”. Diabetes Care. 2005;28:2871.-2876. In the data fusion of method 500, the ANS monitoring is ignored if the blood glucose level is sufficiently high or low, reflecting confidence in the accuracy of the BGL measurement.

If the monitored BGL is not in the specified range (the N option of checking step 501), then in step 505 the system checks whether the BGL is less than or equal to a designated threshold, for example 2.3 mmol/L. If the BGL is below the minimum threshold (the Yes output of step 505) then in step 506 the data fusion module triggers an output alarm. This alarm may be communicated by visual and audio outputs. The alarm may also be used to interrupt or reduce the insulin infused into the patient by the insulin delivery system 58 (FIG. 1).

If the measured BGL is higher than the calibration threshold (for example as a No output of step 505) then the control system 50 proceeds with its standard insulin regime.

The BGL data and ANS data from monitoring steps 512, 514 are also provided to process 200, which is described above with reference to FIG. 4. The output alarm of method 200 (ie the Y output of process 200 as seen in FIG. 5) is provided to the OR block 504. Thus, if the measured BGL is within the specified range and either one of processes 503 and 200 indicates a hypoglycaemic event, then the alarm process 506 is triggered. Other safety monitoring procedures 507 may also provide a safety jacket for the insulin delivery system.

The processing steps of method 500 may be executed on a single processor or in a distributed manner at various locations. Some or all of the processing may, for example, be executed in a CGMS.

Analyses show that the fusion method 500 reduces missed hypoglycaemic events (critical alarms) by over 50% without increasing false alarms overnight.

In other arrangements the threshold check 501 is not a simple threshold test. For example there may be a variable relative weighting between the BGL intermediate alarm and the ANS intermediate alarm. The relative weighting may depend on an expected accuracy of the CGMS in different BGL ranges. Fuzzy logic algorithms may be used to fuse the BGL and ANS data.

Other features or input data may be used to vary the relative effect of the BGL and ANS data in the fusion algorithm 500. For example, the user may enter the result of a finger prick BGL measurement. If this result differs from the CGMS 52 output, greater weight may be placed on the ANS data. Similarly, if anomalies are apparent in the ANS data, for example if the chest-belt unit 2 has been dislodged, then the ANS data may be discounted.

Other forms of data fusion can be derived from the complementary nature of the BGL and ANS data sources. The data fusion enables the implementation of an essential. critical alarm component within closed-loop glycaemic control systems. Specific features of the fusion method may depend on the characteristics of each closed-loop system such as anticipated calibration and drift errors.

In the context of this specification, the word “comprising” or its grammatical variants is equivalent to the term “including” and should not be taken as excluding the presence of other elements or features.

It will be understood that the invention disclosed and defined in this specification extends to all alternative combinations of two or more of the individual features mentioned or evident from the text or drawings. All of these different combinations constitute various alternative aspects of the invention.

1. A system for controlling a flowrate of insulin infused into the body of a patient, the system comprising:
an insulin infusion device that in use infuses insulin into the body of the patient;
a first sensor that in use generates blood glucose level (BGL) data indicative of blood glucose level of the patient;
a second sensor that in use generates autonomic nervous system (ANS) data dependent on at least one parameter of the patient's autonomic nervous system;
a processor that receives the BGL data and the ANS data and, based on the received data, generates an output alarm signal if a hypoglycaemic event is inferred; and
a controller that modifies a flowrate of insulin of the insulin infusion device dependent on the output alarm signal.

2. The system of claim 1 wherein the processor generates a first intermediate alarm signal if the BGL data indicates a hypoglycaemic event.

3. The system of claim 2 wherein the processor generates the first intermediate alarm signal if a measured blood glucose level falls below a specified threshold.

4. The system of claim 1 wherein an intermediate alarm signal is generated if the measured blood glucose level lies within a specified range,

5. The system of claim wherein the ANS data comprises data indicative of a heart rate of the patient.

6. The system of claim 1 wherein the processor analyses time trends of the ANS data, and infers an occurrence of a hypoglycaemic event based on the analysed.

7. The system of claim 6 wherein the processor generates a second intermediate alarm signal if the ANS trend analysis infers the occurrence of a hypoglycaemic event.

8. The system of claim 7 wherein the processor generates the output alarm signal as a function of the first intermediate alarm signal and the second intermediate alarm signal.

9. The system of claim 8 wherein the processor ascribes a relative weighting to the first and second intermediate alarm signals dependent on a blood glucose level measured by the first sensor.

10. The system of claim 9 wherein the relative weighting depends on an expected accuracy of the first sensor in different measurement ranges.

11. The system of claim 7 wherein if the BGL data lies in a euglycaemic range or a hyperglycaemic range the processor decreases the relative weighting of the second intermediate alarm.

12. The system of claim 6 wherein if the BGL data is less than a specified low threshold, the processor outputs the alarm signal irrespective of the second intermediate alarm.

13. The system of claim 12 wherein the low threshold is less than or equal to 2.3 mmol/L.

14. The system of claim 1 wherein the processor analyses time trends of the BGL data and infers an occurrence of a hypoglycaemic event based on the analysed BGL time trends.

15. The system of claim 1 wherein in analysing the time trend of the ANS data the processor utilises one or more parameters that are varied dependent on a measured blood glucose value.

16. The system of claim 4 wherein the specified range is between 2.3 and 4.8 mmol/L.

17. A system for fusing two data sources that are substantially statistically independent, in order to generate an infusion-cut-off signal to control an insulin pump.

18. The system of claim 17 wherein one of the data sources is derived from a continuous glucose monitoring system (CGMS) and the other from autonomic nervous system (ANS) data.

19. A method for monitoring a flowrate of insulin infused into the body of a patient by an insulin infusion device, the method comprising:
receiving blood glucose level (BGL) data indicative of a blood glucose level of the patient;
receiving autonomic nervous system (ANS) data dependent on at least one parameter of the patient's autonomic nervous system;
maintaining an ANS-difference signal data based on a difference between the ANS data and a time-lagged version of the ANS data;
triggering a first intermediate alarm if the BGL data indicates a hypoglycaemic event;
triggering a second intermediate alarm if the ANS-difference signal indicates a hypoglycaemic event; and
outputting an alarm signal to the insulin infusion device if the first intermediate alarm and the second intermediate alarm are triggered.

20. The method of claim 19 wherein the first intermediate alarm is triggered if the BGL data falls below a first specified threshold.

21. The method of claim 19 wherein the first intermediate alarm. is triggered if the BGL data lies within a specified r

22. The method of claim 19, comprising
generating the alarm signal as a function of the first intermediate alarm signal and the second intermediate alarm signal.

23. The method of claim 22 wherein the function comprises a relative weighting of the first intermediate alarm signal and the second intermediate alarm signal, and the method comprises varying the relative weighting depending on the BGL data.

24. The method of claim 23 wherein the relative weighting depends on an expected accuracy of the BGL data in different measurement ranges.

25. The method of claim 24 comprising decreasing the relative weighting of the second intermediate alarm if the BGL data lies in a euglycaemic range or a hyperglycaemic range.

26. The method of claim 19, comprising
outputting an instruction for the insulin infusion device to reduce the flowrate of insulin if the BGL data lies below a specified low threshold.

27. computer program product comprising machine-readable program code recorded on a machine readable recording medium for controlling the operation of a data-processing, apparatus on which the program code executes to perform a method for monitoring a flowrate of insulin infused into the body of a patient by an insulin infusion device, the method comprising:
receiving blood critic (BGL) data indicative of a blood glucose level of the patient;
receiving autonomic nervous system (ANS) data dependent on at least one parameter of the patient's autonomic nervous system;
maintaining an ANS-difference signal data based on a difference between the ANS data and a time-lagged version of the ANS data;
triggering a first intermediate alarm if the BGL data indicates a hypoglycaemic event;
triggering a second intermediate alarm if the ANS-difference signal indicates a hypoglycaemic event; and
outputting an alarm signal to the insulin infusion device if the first intermediate alarm and the second intermediate alarm are triggered.

28. (canceled)
